(19) United States
(12) Patent Application Publication
(10) Pub. No.: US 2004/0167396 A1
(43) Pub. Date: Aug. 26, 2004

(54) Quantitative full aperture tomography imaging system and method

(75) Inventors: David H. Chambers, Livermore, CA (US); Stephen Gerard Azevedo, Livermore, CA (US); Jeffrey Mast, Loveland, CO (US)

Correspondence Address: James S. Tak, Assistant Laboratory Counsel, Lawrence Livermore National Laboratory, P.O. Box 808, L-703, Livermore, CA, 94551, US

(73) Assignee: The Regents of the University of California

(21) Appl. No.: 10/374,570

(22) Filed: Feb. 25, 2003

(51) Int. Cl. A61B 5/05

(52) U.S. Cl. 600/425

(57) ABSTRACT

A quantitative imaging method and system for surrounding a target region with a plurality of transducers such that an acoustic pulse transmitted from one of the transducers may be received as pulse-derived temporal data at the receiving transducers. A controller operably connects to the transducers for selecting different transmission locations around the target region from where acoustic pulses may be transmitted. A signal processor connected to the transducers operates to remove from the received pulse-derived data of each receiving transducer a record of the pulse directly transmitted to the receiving transducer. And the pulse-derived temporal data modified in this manner is used by the data processor to determine preliminary values for a compressibility term and a density term for each point of the target region. The preliminary values for the respective compressibility term and density term obtained from the different transmission locations, are then averaged by the data processor to ultimately produce quantitative image maps of compressibility and density of the target region.

The United States Government has rights in this invention pursuant to Contract No. W-7405-ENG-48 between the United States Department of Energy and the University of California for the operation of Lawrence Livermore National Laboratory.

### FIELD OF THE INVENTION

The present invention relates to ultrasound imaging devices and modalities. More particularly the present invention relates to a full aperture tomography ultrasound imaging system and method for quantitatively imaging a target region to determine compressibility and density of the target.

### BACKGROUND OF THE INVENTION

Ultrasound imaging and tomography has been used as a diagnostic tool in a wide variety of fields, including medicine and industry, e.g. non-destructive testing. Conventional ultrasound systems transmit pulses of high frequency sound into a medium, such as the human body, and map the magnitude of returned echoes. However, these conventional systems only provide images that are proportional to the contrast between the target and the background in which the target resides. Unfortunately, they do not provide insight into the quantitative values of the target, such as the target's compressibility, κ, and density, ρₜ.

One method of determining compressibility and density of a target is disclosed in PCT patent WO 01/01866A1 to Walker, showing an angular imaging system that processes data obtained from multiple scattering angles. The Walker patent, however, utilizes translating transmit and receiver apertures from a transducer array to acquire data at two or more scattering angles, and then processes this data to form images depicting angular scatter information. As shown in FIG. 9, a transmit aperture translator and a receiver aperture translator control the transmit and receiver apertures, respectively, on the transducer array. For each successive pulse transmit, the translators serve to displace the transmit aperture and the receive aperture in equal amounts in opposite directions along a translational axis 5, as shown in FIG. 2(C).

### SUMMARY OF THE INVENTION

One aspect of the present invention includes a method of quantitatively imaging a target region for compressibility and density comprising: (a) surrounding the target region with a plurality of transducers; (b) transmitting an acoustic pulse from one of the transducers to the target region; (c) receiving pulse-derived temporal data at a plurality of the transducers, wherein a transmission location of the acoustic pulse is known relative to the receiving transducers; (d) removing from the received pulse-derived temporal data of each receiving transducer a record of the acoustic pulse directly transmitted thereto, for producing a set of modified pulse-derived temporal data; (e) determining from the set of modified pulse-derived temporal data a preliminary value for each of a compressibility term and a density term for each point of the target region; (f) repeating steps (b) through (e) for different transmission locations encompassing the target region; and (g) averaging the preliminary values of the respective compressibility and density terms obtained from the different transmission locations, to obtain final values thereof for each point of the target region, whereby the final values represent quantitative image maps of the respective compressibility and density terms of the target region.

Another aspect of the present invention includes a quantitative imaging method comprising: (a) surrounding a target region with a transmitter and a plurality of receivers; (b) transmitting an acoustic pulse from the transmitter to the target region, wherein a transmission location of the transmitter is known relative to the receivers; (c) receiving pulse-derived signals at the receivers; (d) pre-processing the received pulse-derived signals of each receiver to remove therefrom a directly transmitted component of the acoustic pulse; (e) determining from the pre-processed pulse-derived signals a preliminary value for each of a compressibility term and a density term for each point of the target region; (f) relocating the transmitter to a different transmission location relative to the target region and repeating steps (b) through (e) for a plurality of different transmission locations encompassing the target region; and (g) averaging the preliminary values of the respective compressibility and density terms obtained from the different transmission locations, to obtain final values thereof for each point of the target region, whereby the final values represent quantitative image maps of the respective compressibility and density terms of the target region.

And another aspect of the present invention includes a quantitative imaging system comprising: a plurality of transducers positionable to surround a target region at known positions relative to each other, with at least one of the transducers capable of transmitting an acoustic pulse toward the target region and a plurality of the transducers, capable of receiving pulse-derived temporal data; a controller operably connected to the plurality of transducers for selecting different transmission locations encompassing the target region to vary the pulse-derived temporal data received at each receiving transducer; a first data processor module for removing from the received pulse-derived temporal data of each receiving transducer a record of the acoustic pulse directly transmitted thereto to produce a set of modified pulse-derived temporal data associated with one of the different transmission locations; a second data processor module for determining from each set of modified pulse-derived temporal data a preliminary value for each of a compressibility term and a density term for each point of the target region; and a third data processor module for averaging the preliminary values of the respective compressibility and density terms obtained from the different transmission locations, to obtain final values thereof for each point of the target region, whereby the final values represent quantitative image maps of the respective compressibility and density terms of the target region.

And another aspect of the present invention includes a quantitative imaging apparatus comprising: a transmitter for transmitting an acoustic pulse toward a target region; a plurality of receivers for receiving pulse-derived temporal data, wherein the transmitter and the plurality of receivers are positionable to surround the target region at known positions relative to each other; a controller for repositioning the transmitter to different transmission locations relative to the target region to vary the pulse-derived temporal data at each receiver; and a data processor adapted to: remove from the pulse-derived temporal data of each receiver a record of the acoustic pulse directly transmitted thereto to produce a set of modified pulse-derived temporal data associated with one of the different transmission locations; determine from each set of modified pulse-derived temporal data a preliminary value for each of a compressibility term and a density term for each point of the target region; and average the preliminary values of the respective compressibility and density terms obtained from the different transmission locations, to obtain final values thereof for each point of the target region, whereby the final values represent quantitative image maps of the respective compressibility and density terms of the target region.

And another aspect of the present invention includes a quantitative imaging system comprising: means for transmitting an acoustic pulse toward a target region from a transmission location; means for receiving pulse-derived signals at various receiving locations surrounding the target region to produce temporal data corresponding to the various receiving locations, wherein the positions of the receiving locations are known relative to the transmitting location; means for changing the transmission location to a plurality of different transmission locations whereby different pulse-derived temporal data may be received at the various receiving locations; first processor means for removing from the pulse-derived temporal data of each receiver a record of the acoustic pulse directly transmitted thereto to produce a set of modified pulse-derived temporal data associated with one of the different transmission locations; second processor means for determining from each set of modified pulse-derived temporal data preliminary values for a compressibility term and a density term for each point on the target region; and third processor means for averaging the preliminary values of the respective compressibility and density terms obtained from the different transmission locations, to obtain final values thereof for each point on the target region, whereby the final values represent quantitative image maps of the respective compressibility and density terms of the target region.

And another aspect of the present invention includes a quantitative imaging system comprising: a plurality of transducers forming a target volume therebetween for receiving a target object to be imaged, with at least one of the transducers capable of transmitting an acoustic pulse into the target volume and a plurality of the transducers capable of receiving pulse-derived temporal data; a controller operably connected to the plurality of transducers for selecting different transmission locations encompassing the target volume to vary the pulse-derived temporal data received at each receiving transducer; a first data processor module for removing from the received pulse-derived temporal data of each receiving transducer a record of the acoustic pulse directly transmitted thereto to produce a set of modified pulse-derived temporal data associated with one of the different transmission locations; a second data processor module for determining from each set of modified pulse-derived temporal data a preliminary value for each of a compressibility term and a density term for each point of a target region; and a third data processor module for averaging the preliminary values of the respective compressibility and density terms obtained from the different transmission locations, to obtain final values thereof for each point of the target region, whereby the final values represent quantitative image maps of the respective compressibility and density terms of the target region.

### BRIEF DESCRIPTION OF THE DRAWINGS

The accompanying drawings, which are incorporated into and form a part of the disclosure, are as follows:

FIG. 1 is a schematic view of a first exemplary operational arrangement of the present invention where a transmitter is translated to different transmission positions.

FIG. 2 is a schematic view of a second exemplary operational arrangement of the present invention where a transmission location is assigned from various transducer positions.

FIG. 3 is a schematic view of an exemplary imaging geometry having a circular configuration and illustrating transmission and reflection angles of the presenting invention.

FIG. 4 is a flow diagram of the process steps in an exemplary embodiment of the present invention.

### DETAILED DESCRIPTION

The present invention is a quantitative imaging system and method incorporating a scattering model into a full aperture tomography (FAT) arrangement and algorithm which enables a beam formed time series to be resolved into a component proportional to compressibility differences and another component proportional to density differences. In particular, a target or target region may be insonified with acoustic ultrasound pulses to obtain quantitative values representing compressibility and density data from reflected and transmitted acoustic signals. Thus the present invention is characterized as a quantitative full aperture tomography (QFAT) system and algorithm. The present invention treats each point in the target region as an isolated scatterer and assumes a weak scattering model, i.e. multiple scattering from one target point to another are not considered. While this assumption simplifies the imaging algorithm, it can introduce artifacts in the image due to multiple scattering events. The present invention serves to minimize these artifacts by various signal processing measures as will be described in detail below.

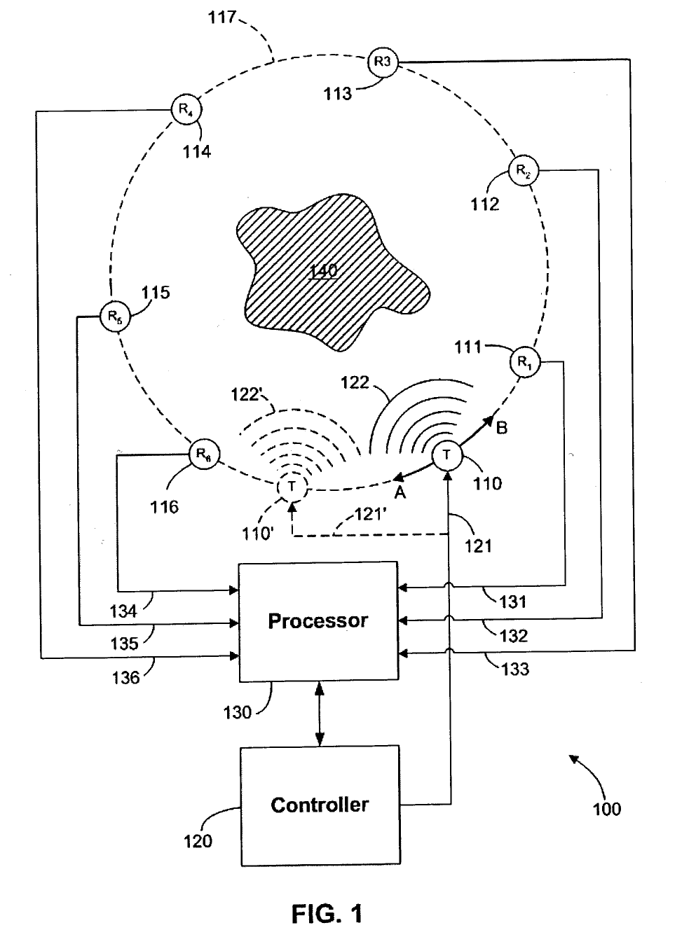

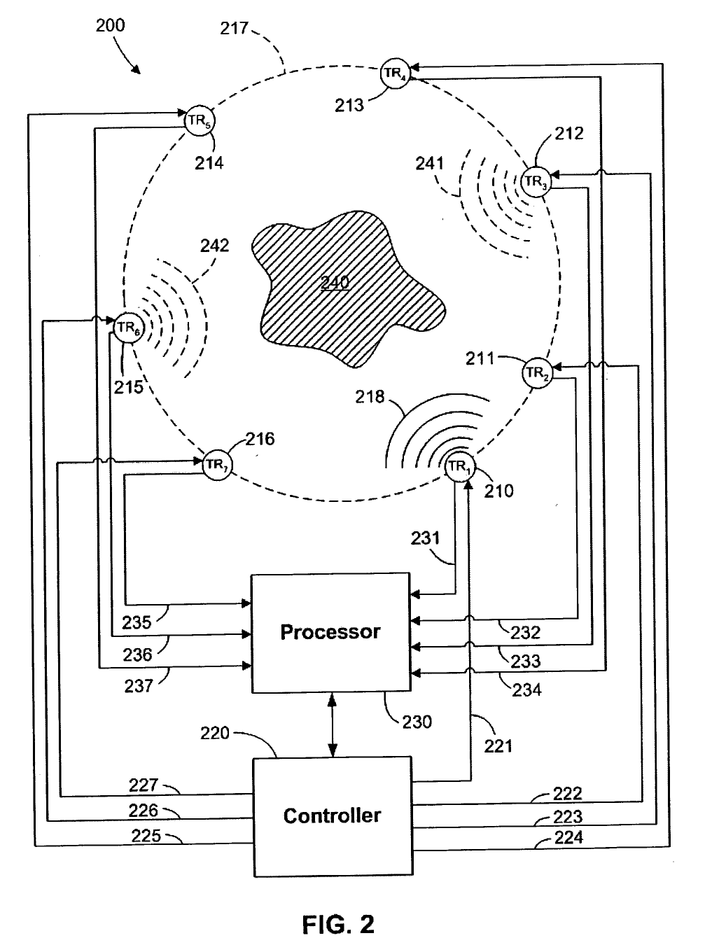

Turning now to the drawings, FIGS. 1 and 2 show two schematic operational arrangements for first and second exemplary imaging systems 100 and 200, respectively, of the present invention. As shown in FIG. 1, the first system 100 includes an acoustic transmitter T indicated at reference character 110 for generating and transmitting an acoustic pulse, and a plurality of acoustic receivers R₁ to R₆ indicated at reference characters 111 to 116, respectively, surrounding a target region 140 for receiving acoustic signals at each receiver as a function of time. The received acoustic signals are derived from the transmitted pulse, and therefore reference hereinafter and in the claims shall be made to “pulse-derived temporal data.” Additionally, the system 100 includes a controller 120, such as a RF unit, operably connected to the transmitter 110 for controlling transmission of an acoustic pulse therefrom. The controller 120 is also adapted to physically relocate the transmitter to different transmission locations. And finally, the system 100 includes a signal/data processor 130 operably connected to the receivers 110-117, and adapted to process the pulse-derived temporal data received at the receivers to ultimately determine compressibility and/or density of the target region 140.

And as shown in FIG. 2, the second system 200 includes a plurality of transducers TR₁ to TR₇ indicated at reference characters 210 to 216, respectively, each of which may be assigned a pulse transmitting and/or signal receiving function. The transducers are arranged surrounding a target region 240 similar to FIG. 1, for receiving pulse-derived temporal data at each receiving transducer as a function of time. Additionally, the system 200 also includes a controller 220 and a processor 230 adapted for similar functions described in FIG. 1. In particular, the controller 220 is operably connected to each of the transducers for assigning a transmission function to a different one of the transducers for successive pulse transmissions, as well as controlling to cause the pulse transmissions. While physical relocation of a transmitting transducer to different transmission locations is not necessary in this arrangement due to the virtual relocation of the transmitter, such functionality is not precluded. And the system 200 includes a signal data processor 230 also operably connected to each transducer 210-217, and adapted to process the pulse-derived temporal data received at the receiving transducers to ultimately determine compressibility and/or density of the target region 240.

Ultrasonic transducers known in the art, e.g. piezoelectric transducers, are used for the transmitter 110 and receivers 111-116 (FIG. 1), and the transducers 210-216 (FIG. 2). And various types of piezoelectric materials may be used for the transducers, such as but not limited to PZT, copolymer, polymer, or composite materials, and preferably having a high electrical-to-mechanical coupling coefficient. Furthermore, a single transducer element or various types of transducer arrays (linear phased array, curved linear array, 2-D array) may be used for each of the transmitter and receivers. It is notable that while operation of the present invention does not depend on any beamforming techniques, such as beam focusing and beam steering upon transmit and receive, such techniques (and the array transducers used thereby) may be optionally incorporated as known in the art for further resolution enhancement. In FIG. 1 of the drawings, transducers having a predetermined function, i.e. transmitting or receiving, are represented with an “R” for receivers (see 111 to 116 in FIG. 1) and “T” for transmitters (110 in FIG. 1). Additionally in FIG. 2, generic transducers which may be assigned either a transmitting or receiving function, as determined by the controller, are represented by “TR.”

As can be seen in FIGS. 1 and 2, the transmitter, receivers, and transducers are positioned to surround the target region with sufficient angular diversity to “see” the entirety of the target region 140, 240 from all sides at a single instance. As used herein and in the claims, the “target region” is any homogeneous or heterogeneous object, region, space, area or volume, which is the subject of inquiry for imaging, including but not limited to medical imaging, and inspection of parts and assemblies. Thus the target region 140 may include a target object in its entirety, or a portion/section thereof. Additionally, the transmitter and receivers may be placed individually or as a unit around an existing target region. Or in the alternative, the transducers may be pre-arranged with respect to each other to define a targeting volume capable of receiving a target object or region therein.

In order to achieve sufficient angular diversity, the transmitter and receivers may be arranged in any number of encompassing geometries, such as the exemplary circular geometries 117 and 217 shown in FIGS. 1 and 2, respectively. It is appreciated, however, that other geometries may also be employed, such as a rectangular, triangular, or even parallel configurations, so long as sufficient angular diversity exists for the receivers to enable viewing of the target or target region from all sides, or as wide an angular range as possible. Moreover, it is appreciated that imaging geometries may also include three-dimensional configurations for surrounding the target region with sufficient angular diversity. For example, the circular configuration shown in FIG. 1 can be representative of a cross-section in a spherical arrangement of transmitter and receivers surrounding the target region 140. In any case, the target-surrounding and encompassing arrangement of the transmitter and receivers illustrated in FIG. 1 produces the “full aperture” of the FAT modality utilized for imaging a target region. This is different from synthetic aperture (SA) techniques, which typically utilize only the part of the scattered field that scatters directly back toward the transmitter, recording the backscatter signal over time to realize a physically non-existent large aperture from the successive use of smaller real apertures. It also differs from common B-scan or medical ultrasound imaging that use beam-steering and focusing techniques on transmit and receive to create images and enhance the signal over noise.

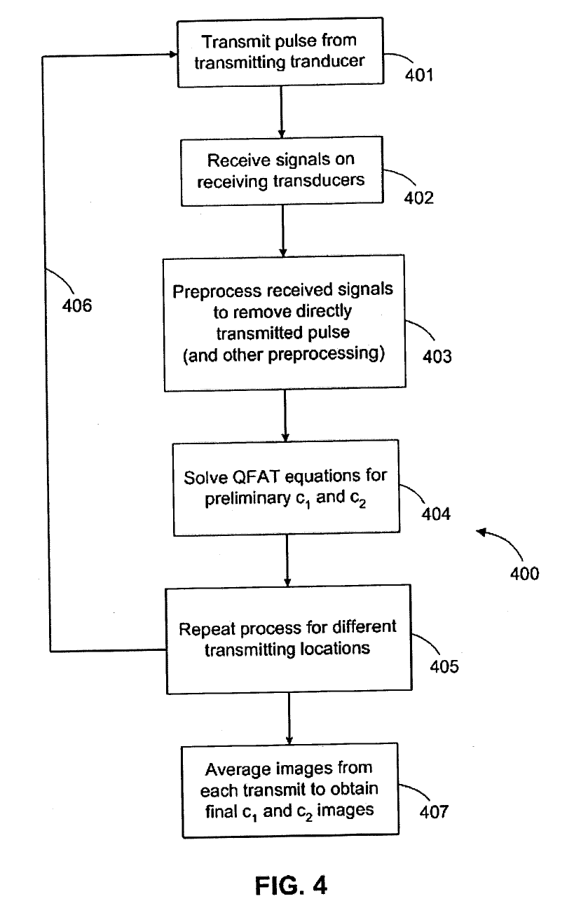

FIG. 4 shows a flowchart 400 of an exemplary algorithm for quantitatively imaging a target region for compressibility and density, according to the present invention. Initially, at step 401, an acoustic pulse is transmitted from a single transmitting transducer toward a target region to insonify the target region. The transmitted pulse is illustrated in FIG. 1 by reference character 122 emanating from transmitter T, at 110, and in FIG. 2 by reference character 218 emanating from transmitting transducer TR₁, at 210. Pulse transmission occurs at a known position relative to the receivers in order to ascertain certain parameters, such as relative distances and reflection angles, necessary for use in subsequent calculations. Pulses may be windowed sine waves, Gaussians, or other kinds of broadband pulses. Resolution improves as the bandwidth of the pulse is increased.

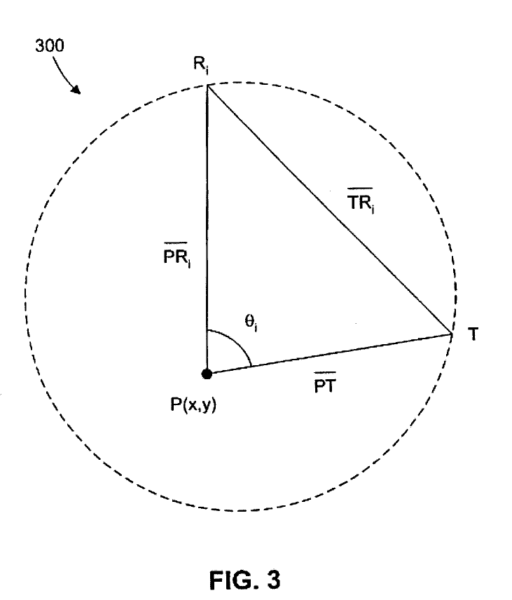

Upon insonifying the target region with a pulse, pulse-derived signals are received at each receiver as indicated at 402 in FIG. 4. The received pulse-derived signals are temporal data represented by the function rᵢ(t) for the i^{th} receiver. The pulse-derived temporal data includes time-delayed scattered echoes produced from within and upon encountering the target region due to changes in acoustic impedance at the interfaces between acoustic media, e.g. different tissue types. Additionally, the pulse-derived temporal data includes a directly transmitted component of the transmitted pulse traveling directly from the transmitter to a receiver. A representative pulse transmission and signal reception geometry 300 is shown in FIG. 3 illustrating these two components of the pulse-derived temporal data. In particular, FIG. 3 shows the reflected and direct transmission trajectories and reflection angle of a transmitted pulse, as well as the distances between a representative scattering point, P(x,y) on the target region, a given receiver, Rᵢ, and the transmitter, T. Additionally, the reflection angle θᵢ is also shown produced by reflection from point P(x,y).

Upon receiving all pulse-derived temporal data at the receivers, the processor 130, 230 performs calculations to determine the compressibility and density values for each point of the target region. In particular, at step 403 in FIG. 4, the received pulse-derived temporal data is initially preprocessed by the processor to remove a record of the transmitted pulse that travels directly to the receiver without experiencing any scattering. In FIG. 3 a representative directly-transmitted pulse from the transmitter T to the receiver R is shown as the line segment TRᵢ. This pulse component can be several orders of magnitude larger in the received data than the scattered energy from the point targets. Thus removal of this component serves to sharpen the images and improve the resolution of the quantitative results. The direct pulse component is removed by setting rᵢ(t)=0 for t<t_{d}+tₚ where t_{d} is the pulse travel time between the transmitter and receiver, and tₚ is the transmitter pulse length in time. The estimate for t_{d} is made assuming an average propagation velocity, ν, for the target region. It is notable that the processor is typically a CPU, or electronic circuitry configured to perform calculations and/or other specified functions. Moreover, the processor may comprise independent processor modules configured to perform specific processing and/or preprocessing operations on the data.

Furthermore, additional pre-processing steps may be employed by the processor to further sharpen images and improve the resolution of the quantitative results. For example, a deconvolution computation may be performed on the received data r(t) to remove the transmitted pulse spread and concentrate the energy in time from a given point scatterer. The deconvolved result is represented by the equation:

$${r}_{i}^{′} ( t ) = {IFT}_{f} ( \frac{{r}_{i} ( f )}{p ( f ) + σ} )$$

where IFT_{f} represents the inverse Fourier transform with respect to f, rᵢ(f) is the Fourier transform of rᵢ(t), and p(f) is the Fourier transform of the transmitted pulse, p(t). The regularization parameter, σ, prevents division by zero when the pulse spectrum goes to zero.

Another exemplary preprocessing step is to generate rᵢ(t) as an analytic signal by zeroing out the negative frequency components of rᵢ(f) before performing the inverse Fourier transform. Using the analytic signal reduces the effects of superpositions of scattered energy from scatters other than the one of interest since they tend to cancel during the QFAT calculation. This step helps to reduce artifacts from multiple scattering events and clutter in the final image. It is appreciated that while these and other supplemental preprocessing steps are not required to implement the present invention, they can serve to further enhance resolution. Other preprocessing options include various filtering and smoothing operations known in the relevant art to reduce noise. In addition, the envelope of each time signal in the data could be extracted and used in place of the actual time signal in subsequent steps.

Completion of the preprocessing steps enables the now modified pulse-derived temporal data to be used in determining the compressibility κₜ and density ρₜ of each point of the target region. This is accomplished by first determining preliminary values for a compressibility term c₁ and a density term c₂ for each point in the region of interest, i.e. the target region. And as indicated at step 404 of FIG. 4, the preliminary values for c₁ and c₂ are determined using QFAT equations, derived as follows.

As discussed above, each of the receivers detect pulse-derived signals which are temporal data, i.e. time-delayed due to the various paths traveled by such signals. Based on acoustic wave theory, the contributed scattered field amplitude (or its envelope) from a point, P(x,y), in the target region due to the pulse insonification from transmitter, T, is represented at the i^{th} receiver as follows:

rᵢ(t_{d})=c₁+c₂ cos(θᵢ)

where

$${t}_{d} = \frac{\overline{PR} + \overline{PT}}{v} , {c}_{1} = \frac{{κ}_{t} + κ}{κ} , {c}_{2} = \frac{3 {ρ}_{t} - 3 ρ}{2 {ρ}_{t} + ρ} and$$

$$cos ( θ ) = \frac{{\overline{{PR}_{i}}}^{2} + {\overline{PT}}^{2} - {\overline{{TR}_{i}}}^{2}}{2 \overline{{PR}_{i}} * \overline{PT}} .$$

In the above equations, κ and ρ are the compressibility and density, respectively, of the background material.

For N number of receivers, the system obtains N linear equations representing the data recorded at each of the receivers due to the scattering from the target point P(x,y):

$${c}_{1} + {c}_{2} {w}_{1} = {r}_{1}$$

$$⋮$$

$${c}_{1} + {c}_{2} {w}_{N} = {r}_{N}$$

where wᵢ represents cos(θᵢ) and rᵢ represents rᵣ(t_{d}). Rewriting these equations in matrix form gives:

$$[ \begin{matrix}1 & {w}_{1} \\ ⋮ & ⋮ \\ 1 & {w}_{N}\end{matrix} ] [ \begin{matrix}{c}_{1} \\ {c}_{2}\end{matrix} ] = [ \begin{matrix}{r}_{1} \\ ⋮ \\ {r}_{N}\end{matrix} ]$$

These equations are now in the form

A\overline{x}=\overline{b}

where

$$A = [ \begin{matrix}1 & {w}_{1} \\ ⋮ & ⋮ \\ 1 & {w}_{N}\end{matrix} ] , \overline{x} = [ \begin{matrix}{c}_{1} \\ {c}_{2}\end{matrix} ] , and \overline{b} = [ \begin{matrix}{r}_{1} \\ ⋮ \\ {r}_{N}\end{matrix} ] .$$

In this form, preliminary values for c₁ and c₂ can be determined using the least mean square (LMS) solution

\overline{x}=(A^{T}A)⁻¹A^{T}\overline{b}

with

$${A}^{T} A = [ \begin{matrix}N & \sum {w}_{i} \\ \sum {w}_{i} & \sum {w}_{i}^{2}\end{matrix} ] , {( {A}^{T} A )}^{- 1} = \frac{1}{d} [ \begin{matrix}\sum {w}_{i}^{2} & - \sum {w}_{i} \\ - \sum {w}_{i} & N\end{matrix} ] , \\ {A}^{T} b = [ \begin{matrix}\sum {r}_{i} \\ \sum {r}_{i} {w}_{i}\end{matrix} ] , and$$

d=NΣwᵢ²−(Σwᵢ)². Substituting and solving results in the QFAT equations:

$$QFAT = [ \begin{matrix}{c}_{1} \\ {c}_{2}\end{matrix} ] = \frac{1}{d} [ \begin{matrix}\sum {r}_{i} \sum {w}_{i}^{2} - \sum {r}_{i} {w}_{i} \sum {w}_{i} \\ N \sum {r}_{i} {w}_{i} - \sum {r}_{i} \sum {w}_{i}\end{matrix} ] .$$

Quantitative image maps of the preliminary values for the compressibility term, c₁, and the density term, c₂, may thus be obtained throughout the target region by performing the preceding computation for each point P(x,y) in the target space via the processor.

At step 405 of FIG. 4, the aforementioned data acquisition steps (beginning with pulse transmission of 401) are iteratively repeated for a desired or predetermined number of different transmission locations around the target region. Two illustrative methods for relocating the transmission location are shown in FIGS. 1 and 2.

In FIG. 1, different transmission locations are realized by physically actuating, translating, or otherwise moving the designated transmitter 110 relative to the target region 140. In particular, the transmitter T 110 is rotatably translated in the direction of arrow A to a new position along the circle 117 indicated at reference character 110′, where a second pulse 122′ may be transmitted. Actuation of the transmitter 110 is effected by the controller 120 which is operably connected thereto. While FIG. 1 shows the relocation of the transmitter following a prescribed geometric path as indicated by arrows A and B, it is not limited only to such. Rather, physical relocation of the transmitter may be effected in any suitable manner known in the art, and along any suitable path so long as the new transmission location is known to all receivers. It is notable that while FIG. 1 shows the relocation of the transmitter T alone, the receivers R may also be simultaneously repositioned together with the transmitter, such as when the transmitter and receivers have a monolithic construction.

As can be seen in FIG. 2, different transmission locations may also be realized by reassigning a transmission function to a different one of the transducers TR₁ to TR₇ for each pulse transmission. In such a case, each of the transducers is capable of both receiving and transmitting acoustic signals, and the controller determines a transmission or receiving function, or both for each transducer for each transmission. In this manner the transducers may remain physically stationary, while the functionality of at least one of the transducers is selectively varied. For example, prior to the transmission of a first pulse 218 from TR₁, at 210, the controller 220 assigns a transmitting (and possibly receiving) function for TR₁, while the remaining transducers TR₂ to TR₇ are assigned a receiving function only. Next, prior to the transmission of a second pulse 241 from transducer TR₃ 212 at a second location, the controller reassigns the function of TR1 to only a receiving function while TR3 is now assigned a transmitting (and possibly receiving) function. And finally a third illustrative pulse transmission 242 is shown at a third different transmission location of transducer TR₆ 215. It is appreciated that for every pulse transmit, the transducers are held stationary at known distances relative to each other.

After transmitting pulses from multiple transmission locations, final estimates may be obtained for the compressibility term c₁ and the density term c₂ by averaging the results from each transmit, as indicated at step 407 of FIG. 4. This averaging is equivalent to compound imaging and reduces the effect of speckle noise and other artifacts such as shadowing that are common in imaging algorithms using data from limited viewpoints. The final quantitative image values of c₁ and c₂ can be converted to quantitative images of density and compressibility (or sound speed) of the target region given the density ρ and compressibility κ (or sound speed) of the background material:

$${κ}_{t} = κ ( 1 + {c}_{1} ) , \qquad {ρ}_{t} = \frac{3 + {c}_{2}}{3 - 2 {c}_{2}} ρ .$$

It is notable that further image processing may be applied, e.g. smoothing, filtering, to improve the appearance or highlight particular features of interest. Furthermore, while not shown in the drawings, the quantitative results may be displayed as graphic images on a monitor for viewing by a user. In this regard, the signal processor of the present invention may also include an image processing module for generating such graphic images from the processed pulse-derived temporal data.

While particular operational sequences, materials, temperatures, parameters, and particular embodiments have been described and or illustrated, such are not intended to be limiting. Modifications and changes may become apparent to those skilled in the art, and it is intended that the invention be limited only by the scope of the appended claims.

We claim:

1. A method of quantitatively imaging a target region for compressibility and density comprising:
(a) surrounding the target region with a plurality of transducers;
(b) transmitting an acoustic pulse from one of the transducers to the target region;
(c) receiving pulse-derived temporal data at a plurality of the transducers, wherein a transmission location of the acoustic pulse is known relative to the receiving transducers;
(d) removing from the received pulse-derived temporal data of each receiving transducer a record of the acoustic pulse directly transmitted thereto, for producing a set of modified pulse-derived temporal data;
(e) determining from the set of modified pulse-derived temporal data a preliminary value for each of a compressibility term and a density term for each point of the target region;
(f) repeating steps (b) through (e) for different transmission locations encompassing the target region; and
(g) averaging the preliminary values of the respective compressibility and density terms obtained from the different transmission locations, to obtain final values thereof for each point of the target region, whereby the final values represent quantitative image maps of the respective compressibility and density terms of the target region.

2. The method of claim 1,
wherein the preliminary values c₁ and c₂ for the compressibility term and the density term, respectively, for each point of the target region are determined using a least mean square solution represented by the equation:
$$[ \begin{matrix}{c}_{1} \\ {c}_{2}\end{matrix} ] = \frac{1}{d} [ \begin{matrix}\sum {r}_{i} \sum {w}_{i}^{2} - \sum {r}_{i} {w}_{i} \sum {w}_{i} \\ N \sum {r}_{i} {w}_{i} - \sum {r}_{i} \sum {w}_{i}\end{matrix} ] .$$

3. The method of claim 1,
further comprising deconvolving the received pulse-derived data to improve resolution, wherein the deconvolved result is represented by the equation:
$${r}_{i}^{′} ( t ) = {IFT}_{f} ( \frac{{r}_{i} ( f )}{p ( f ) + σ} ) .$$

4. The method of claim 3,
further comprising zeroing out the negative frequency components of rᵢ(f) prior to performing the inverse Fourier transform, for reducing artifacts and clutter in the quantitative image maps.

5. The method of claim 1,
further comprising the step of converting the respective quantitative image maps of the compressibility term c₁ and the density term c₂ into corresponding quantitative image maps of compressibility κₜ and density ρₜ of the target region represented by the equations:
$${κ}_{t} = κ ( 1 + {c}_{1} ) , {ρ}_{t} = \frac{3 + {c}_{2}}{3 - 2 {c}_{2}} ρ .$$

6. The method of claim 1,
wherein the different transmission locations are selected from the transducer locations by assigning a transmission function to a different one of the transducers for each pulse transmission.

7. The method of claim 1,
wherein the different transmission locations are selected by relocating the transmitting transducer for each pulse transmission.

8. A quantitative imaging method comprising:
(a) surrounding a target region with a transmitter and a plurality of receivers;
(b) transmitting an acoustic pulse from the transmitter to the target region, wherein a transmission location of the transmitter is known relative to the receivers;
(c) receiving pulse-derived signals at the receivers;
(d) pre-processing the received pulse-derived signals of each receiver to remove therefrom a directly transmitted component of the acoustic pulse;
(e) determining from the pre-processed pulse-derived signals a preliminary value for each of a compressibility term and a density term for each point of the target region;
(f) relocating the transmitter to a different transmission location relative to the target region and repeating steps (b) through (e) for a plurality of different transmission locations encompassing the target region; and
(g) averaging the preliminary values of the respective compressibility and density terms obtained from the different transmission locations, to obtain final values thereof for each point of the target region, whereby the final values represent quantitative image maps of the respective compressibility and density terms of the target region.

9. The method of claim 8,
wherein the preliminary values c₁ and c₂ for the compressibility term and the density term, respectively, for each point of the target region are determined using a least mean square solution represented by the equation:
$$[ \begin{matrix}{c}_{1} \\ {c}_{2}\end{matrix} ] = \frac{1}{d} [ \begin{matrix}\sum {r}_{i} \sum {w}_{i}^{2} - \sum {r}_{i} {w}_{i} \sum {w}_{i} \\ N \sum {r}_{i} {w}_{i} - \sum {r}_{i} \sum {w}_{i}\end{matrix} ] .$$

10. The method of claim 8,
wherein the preprocessing step includes deconvolving the received pulse-derived data to improve resolution, wherein the deconvolved result is represented by the equation:
$${r}_{i}^{′} ( t ) = {IFT}_{f} ( \frac{{r}_{i} ( f )}{p ( f ) + σ} ) .$$

11. The method of claim 10,
wherein the preprocessing step includes zeroing out the negative frequency components of rᵢ(f) prior to performing the inverse Fourier transform, for reducing artifacts and clutter in the quantitative image maps.

12. The method of claim 8,
further comprising the step of converting the respective quantitative image maps of the compressibility term c₁ and the density term c₂ into corresponding quantitative image maps of compressibility κₜ and density ρₜ of the target region represented by the equations:
$${κ}_{t} = κ ( 1 + {c}_{1} ) , {ρ}_{t} = \frac{3 + {c}_{2}}{3 - 2 {c}_{2}} ρ .$$

13. The method of claim 8,
wherein the plurality of receivers are fixed with respect to the transmitter whereby relocation of the transmitter simultaneously relocates the receivers.

14. A quantitative imaging system comprising:
a plurality of transducers positionable to surround a target region at known positions relative to each other, with at least one of the transducers capable of transmitting an acoustic pulse toward the target region and a plurality of the transducers capable of receiving pulse-derived temporal data;
a controller operably connected to the plurality of transducers for selecting different transmission locations encompassing the target region to vary the pulse-derived temporal data received at each receiving transducer;
a first data processor module for removing from the received pulse-derived temporal data of each receiving transducer a record of the acoustic pulse directly transmitted thereto to produce a set of modified pulse-derived temporal data associated with one of the different transmission locations;
a second data processor module for determining from each set of modified pulse-derived temporal data a preliminary value for each of a compressibility term and a density term for each point of the target region; and
a third data processor module for averaging the preliminary values of the respective compressibility and density terms obtained from the different transmission locations, to obtain final values thereof for each point of the target region, whereby the final values represent quantitative image maps of the respective compressibility and density terms of the target region.

15. The system of claim 14,
wherein the controller is adapted to select the different transmission locations by actuating a transmitting transducer to the different transmission locations.

16. The system of claim 14,
wherein the controller is adapted to select the different transmission locations from the transducer locations by assigning a transmission function to a different one of the transducers for each pulse transmission.

17. The system of claim 14,
wherein the transducers are each capable of transmitting an acoustic pulse and receiving pulse-derived temporal data.

18. The system of claim 14,
wherein the second data processor module is adapted to determine the preliminary values c₁ and c₂ for the compressibility term and the density term, respectively, for each point of the target region are determined using a least mean square solution represented by the equation:
$$[ \begin{matrix}{c}_{1} \\ {c}_{2}\end{matrix} ] = \frac{1}{d} [ \begin{matrix}\sum {r}_{i} \sum {w}_{i}^{2} - \sum {r}_{i} {w}_{i} \sum {w}_{i} \\ N \sum {r}_{i} {w}_{i} - \sum {r}_{i} \sum {w}_{i}\end{matrix} ] .$$

19. The system of claim 14,
further comprising a fourth data processor module adapted to deconvolve the received pulse-derived data to improve resolution, according to the equation:
$${r}_{i}^{′} ( t ) = {IFT}_{f} ( \frac{{r}_{i} ( f )}{p ( f ) + σ} ) .$$

20. The system of claim 19,
further comprising a fifth data processor module adapted to zero out the negative frequency components of rᵢ(f) prior to performing the inverse Fourier transform, for reducing artifacts and clutter in the quantitative image maps.

21. The system of claim 14,
further comprising a sixth data processor module for converting the respective quantitative image maps of the compressibility term c₁ and the density term c₂ into corresponding quantitative image maps of compressibility κₜ and density ρₜ of the target region represented by the equations:
$${κ}_{t} = κ ( 1 + {c}_{1} ) , {ρ}_{t} = \frac{3 + {c}_{2}}{3 - 2 {c}_{2}} ρ .$$

22. A quantitative imaging apparatus comprising:
a transmitter for transmitting an acoustic pulse toward a target region;
a plurality of receivers for receiving pulse-derived temporal data, wherein the transmitter and the plurality of receivers are positionable to surround the target region at known positions relative to each other;
a controller for repositioning the transmitter to different transmission locations relative to the target region to vary the pulse-derived temporal data at each receiver; and
a data processor adapted to: remove from the pulse-derived temporal data of each receiver a record of the acoustic pulse directly transmitted thereto to produce a set of modified pulse-derived temporal data associated with one of the different transmission locations; determine from each set of modified pulse-derived temporal data a preliminary value for each of a compressibility term and a density term for each point of the target region; and average the preliminary values of the respective compressibility and density terms obtained from the different transmission locations, to obtain final values thereof for each point of the target region, whereby the final values represent quantitative image maps of the respective compressibility and density terms of the target region.

23. The apparatus of claim 22,
wherein the plurality of receivers are fixed with respect to the transmitter, whereby the controller simultaneously relocates the plurality of receivers together with the transmitter.

24. A quantitative imaging system comprising:
means for transmitting an acoustic pulse toward a target region from a transmission location;
means for receiving pulse-derived signals at various receiving locations surrounding the target region to produce temporal data corresponding to the various receiving locations, wherein the positions of the receiving locations are known relative to the transmitting location;
means for changing the transmission location to a plurality of different transmission locations whereby different pulse-derived temporal data may be received at the various receiving locations;
first processor means for removing from the pulse-derived temporal data of each receiver a record of the acoustic pulse directly transmitted thereto to produce a set of modified pulse-derived temporal data associated with one of the different transmission locations;
second processor means for determining from each set of modified pulse-derived temporal data preliminary values for a compressibility term and a density term for each point on the target region; and
third processor means for averaging the preliminary values of the respective compressibility and density terms obtained from the different transmission locations, to obtain final values thereof for each point on the target region, whereby the final values represent quantitative image maps of the respective compressibility and density terms of the target region.

25. A quantitative imaging system comprising:
a plurality of transducers forming a target volume therebetween for receiving a target object to be imaged, with at least one of the transducers capable of transmitting an acoustic pulse into the target volume and a plurality of the transducers capable of receiving pulse-derived temporal data;
a controller operably connected to the plurality of transducers for selecting different transmission locations encompassing the target volume to vary the pulse-derived temporal data received at each receiving transducer;
a first data processor module for removing from the received pulse-derived temporal data of each receiving transducer a record of the acoustic pulse directly transmitted thereto to produce a set of modified pulse-derived temporal data associated with one of the different transmission locations;
a second data processor module for determining from each set of modified pulse-derived temporal data a preliminary value for each of a compressibility term and a density term for each point of a target region; and
a third data processor module for averaging the preliminary values of the respective compressibility and density terms obtained from the different transmission locations, to obtain final values thereof for each point of the target region, whereby the final values represent quantitative image maps of the respective compressibility and density terms of the target region.
